(19) United States
(12) Patent Application Publication
(10) Pub. No.: US 2012/0277808 A1
(43) Pub. Date: Nov. 1, 2012

(54) INSTRUMENT AND METHOD FOR REDUCING ELONGATE CONNECTING ELEMENTS

(75) Inventor: Jason Michael May, Cordova, TN (US)

(73) Assignee: Warsaw Orthopedic, Inc., Warsaw, IN (US)

(21) Appl. No.: 13/093,909

(22) Filed: Apr. 26, 2011

(51) Int. Cl. A61B 17/58

(52) U.S. Cl. 606/86 A

(57) ABSTRACT

One non-limiting embodiment is directed to a reduction instrument releasably engageable with a bone anchor and structured to reduce a connecting element into a receiving member of the bone anchor. The reduction instrument includes a first member and a second member laterally movable relative to the first member between a first position for receiving a portion of the bone anchor and a second position for engaging the reduction instrument with the bone anchor. The reduction instrument also includes an actuating member axially displaceable relative to the first and second members. Additionally, the second member is moved between the first and second positions upon displacement of the actuating member relative to the first and second members, and the second member extends substantially parallel to the first member as it is moved between the first and second positions.

### BACKGROUND

The present invention relates to a surgical instrument and a manner of using the same, and more particularly but not exclusively relates to the reduction of elongate connecting elements or implant components, such as spinal rods, to one or more bone anchors in an orthopedic construct for treatment of a spinal deformity.

The use of surgical instruments to place components in orthopedic constructs has become commonplace. In particular, spinal implant systems frequently include several bone anchors and an interconnecting rod shaped to provide a desired spinal curvature. Typically, the bone anchors are implanted first and the rod is then fixed to the bone anchors in succession. As this procedure progresses, some degree of force may need to be applied to reduce the distance between the rod and the next anchor to be connected to it. Accordingly, various instruments have been described to facilitate such rod reduction. In this arena, the desire persists for improved rod reducing capability. Thus, there is a need for additional contributions in this area of technology.

### SUMMARY

One nonlimiting embodiment of the present invention is directed to a surgical instrument releasably engageable with a bone anchor and structured to reduce a connecting element into a receiving member of the bone anchor. The instrument includes a first member and a second member laterally movable relative to the first member between a first position for receiving a portion of the bone anchor and a second position for engaging the reduction instrument with the bone anchor. The instrument also includes an actuating member axially displaceable relative to the first and second members. Additionally, the second member is moved between the first and second positions upon displacement of the actuation member relative to the first and second members, and the second member extends substantially parallel to the first member as it is moved between the first and second positions. However, in other embodiments, different forms and applications are envisioned.

Another embodiment of the present invention is directed to a reduction instrument that includes a first member extending between a proximal end and an opposite distal end including a first anchor engagement portion. The instrument also includes a second member extending between a proximal end and an opposite distal end including a second anchor engagement portion. The second member is laterally movable relative to the first member between a first position spaced apart from the first member and a second position between the first position and the first member. The instrument also includes an actuation member axially displaceable relative to the first and second members. Additionally, the second member is moved between the first and second positions upon displacement of the actuation member relative to the first and second members, and the second member extends substantially parallel to the first member during movement between the first and second positions.

Still, another embodiment of the present invention is directed to a reduction instrument that includes a first member extending between a proximal end and an opposite distal end including a first anchor engagement portion, and a second member extending between a proximal end and an opposite distal end including a second anchor engagement portion. A first linkage assembly is pivotably coupled with the first and second members and is laterally adjustable between a first configuration where the second member is in a first position spaced apart from the first member, and a second configuration where the second member is in a second position between the first position and the first member. The instrument also includes an actuation member axially displaceable relative to the first and second members, and the first linkage assembly is moved between the first and second configurations upon displacement of the actuation member relative to the first and second members.

A further embodiment of the present invention is directed to a method that includes providing an anchor having a bone engaging portion and a receiver member having a receiving portion, and providing a reduction instrument engageable with the anchor. The reduction instrument includes a first member extending between a proximal end and an opposite distal end including a first anchor engagement portion, a second member extending between a proximal end and an opposite distal end including a second anchor engagement portion, and an actuation member axially displaceable relative to the first and second members. The method also includes positioning the reduction instrument adjacent the anchor, and distally moving the actuation member relative to the first and second members toward the anchor to displace the second member toward the first member and releasably couple the reduction instrument with the anchor, with the second member extending substantially parallel to the first member as the second member is displaced toward the first member.

Another embodiment of the present invention is directed to a unique instrument for surgery in a patient. Other embodiments include unique instruments, methods, systems, devices, kits, assemblies, equipment, and/or apparatus involving the reduction of an elongate connecting element.

Further embodiments, forms, features, aspects, benefits, objects and advantages of the present invention shall become apparent from the detailed description and figures provided herewith.

### BRIEF DESCRIPTION OF THE FIGURES

FIG. 1 is a posterior view of a spinal column segment and a spinal implant system attached thereto.

FIG. 2 is a perspective view of a surgical reduction instrument for positioning an elongate connecting element in a desired position relative to an anchor.

FIG. 3 is a perspective view of an anchor engaging portion of the instrument illustrated in FIG. 2.

FIG. 4 is an exploded assembly view of the anchor engaging portion illustrated in FIG. 3.

FIG. 5 is a front view of the instrument illustrated in FIG. 2 in an anchor receiving configuration.

FIG. 6 is a side view of the instrument illustrated in FIG. 5.

FIG. 7 is a front view of the instrument illustrated in FIG. 2 in an anchor engaging configuration.

FIG. 8 is a side view of the instrument illustrated in FIG. 7.

FIG. 9 is a perspective view illustrating the use of the instrument of FIG. 2 for reducing an elongate connecting element into a receiving portion of an anchor.

FIG. 10 is another perspective view illustrating the use of the instrument of FIG. 2 for reducing an elongate connecting element into a receiving portion of an anchor.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices and described methods, and any such further applications of the principles of the invention as illustrated herein are contemplated as would normally occur to one skilled in the art to which the invention relates.

The present invention is generally directed to a surgical instrument releasably engageable with a bone anchor and structured to reduce a connecting element into a receiving member of the bone anchor. The instrument includes a first member and a second member laterally movable relative to the first member between a first position for receiving a portion of the bone anchor and a second position for engaging the reduction instrument with the bone anchor. The instrument also includes an actuating member axially displaceable relative to the first and second members. Additionally, the second member is moved between the first and second positions upon displacement of the actuating member relative to the first and second members, and the second member extends substantially parallel to the first member as it is moved between the first and second positions. In one form, the elongate connecting element is a spinal rod and the instrument is configured to reduce the spinal rod into one or more receiver members of bone anchors engaged with one or more vertebrae in order to provide support and stabilization of the vertebra(e). However, use of the instrument in other surgical procedures and in connection with anatomical locations other than the spinal column is also contemplated.

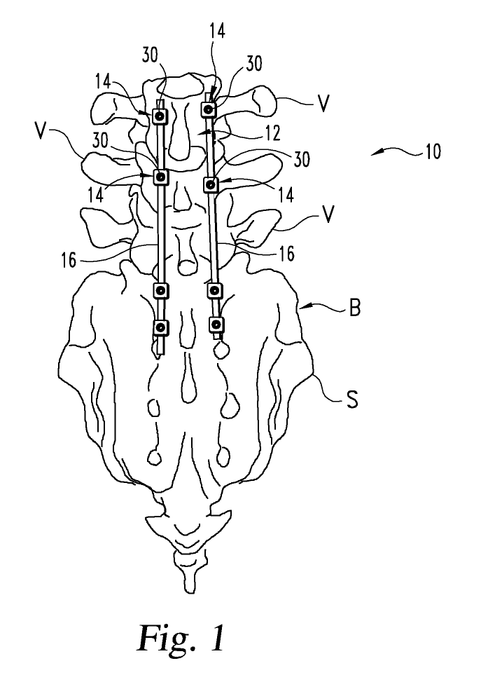

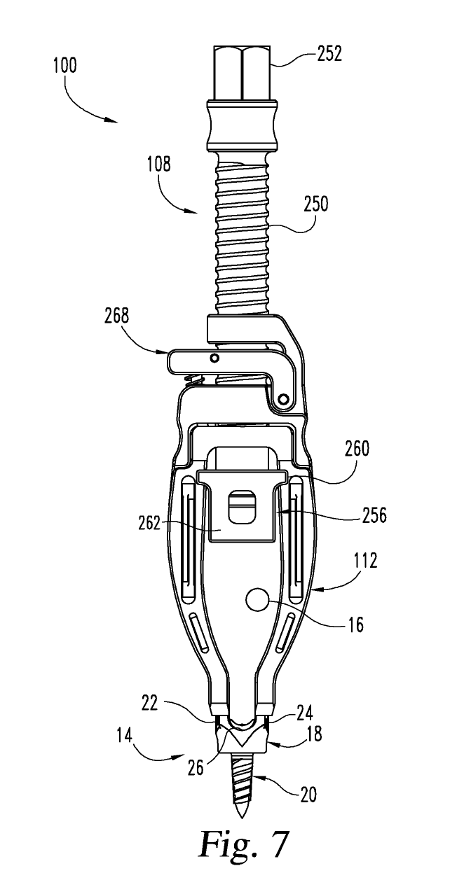

One non-limiting form of a spinal implant system 10 located along a spinal column of a patient and with which the instrument of the present invention may be used is illustrated in FIG. 1. More specifically, in the illustrated form, the implant system 10 is affixed to bones B of the spinal column segment 12 from a posterior approach, although it should be appreciated that implant systems affixed from other surgical approaches to the spine, including anterior, posterior, posterior mid-line, lateral, postero-lateral, and/or antero-lateral approaches, are also contemplated. Bones B include the sacrum S and several vertebrae V. The implant system 10 generally includes several bone anchors 14 and elongate connecting elements 16 structured to selectively interconnect the bone anchors 14. As illustrated in FIGS. 7-10, the bone anchor 14 includes a receiver member 18 configured to receive the connecting element 16 and a distal bone engaging portion 20. In the illustrated embodiment, the bone engaging portion 20 is a bone screw with a threaded shank structured to engage with the bony structure of an underlying vertebra V when the bone anchor 14 is engaged to the spinal column. The receiver member 18 includes a pair of opposing branches 22, 24 defining a channel 26 extending between and opening through opposite first and second sides of the receiver member 18. The branches 22, 24 further define a proximal/distally extending opening that opens through a proximal end of the receiver member 18 to receive a set screw 30 structured to engage with internal threading 28 on the branches 22, 24 to secure the connecting element 16 in the channel 26. While not illustrated, it should be appreciated that the branch 22 includes a pair of laterally facing receptacles oppositely positioned on the first and second sides of the receiver member 18, and that the branch 24 also includes a pair of laterally facing receptacles oppositely positioned on the first and second sides of the receiver member 18.

In one form, the bone engaging portion 20 is pivotally received in the receiver member 16 through a distal opening thereof, and structured to interact therewith to provide the anchor 14 with multi-axial capabilities that permit either a selected number of positions or an infinite number of positions of the bone engaging portion 20 relative to the receiver member 18. Other forms for the anchor 14 are contemplated, including uni-axial and uni-planar configurations. The bone engaging portion 20 can also be in the form of a spike, staple, hook, fusion device, cannulated screw, fenestrated screw, interbody device, intrabody device, clamp, plate, suture anchor, bolt, pin or other bone engaging configurations. The receiving portion 18 can be in the form of a saddle, yoke, eye-bolt or through-hole, side opening member, bottom opening member, top-opening member, eyelet, or any other structure engageable to the connecting element 16 and the instruments disclosed herein.

In the illustrated embodiment, the connecting element 16 is a rigid spinal rod having a substantially linear configuration between its ends. However, it is contemplated that the connecting element 16 can have a curvature that extends along a constant arc, a varying arc, and/or is compounded between its ends. Additionally, in other forms, the connecting element 16 can include any known configuration for a rod, implant or fastener. It is further contemplated that the connecting element 16 can be non-rigid, elastic and/or super-elastic and in the form of a cable, band, wire, or artificial ligament and may be used in tethering or guiding, and/or in other surgical procedures.

In the implant system 10, the bone anchors 14 are affixed to various locations of the spinal column 12 and interconnected with the connecting elements 16. While the illustrated form of the implant system 10 includes bilaterally positioned connecting elements 16, it should be appreciated that forms in which a single connecting element 16 is positioned on a single side of the spinal column 12 are also possible. Additionally, while not previously discussed, it should be appreciated that the spinal implant system 10 may be used for, but is not limited to, treatment of degenerative spondylolisthesis, fracture, dislocation, scoliosis, kyphosis, spinal tumor and/or a failed previous fusion.

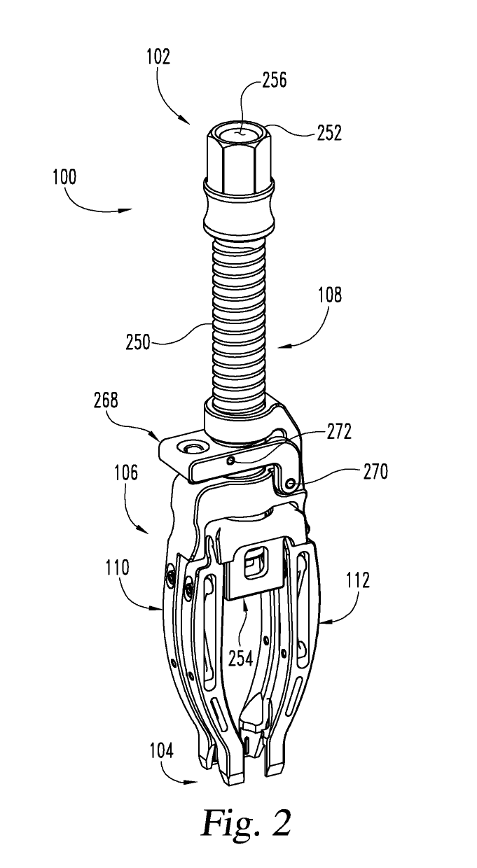

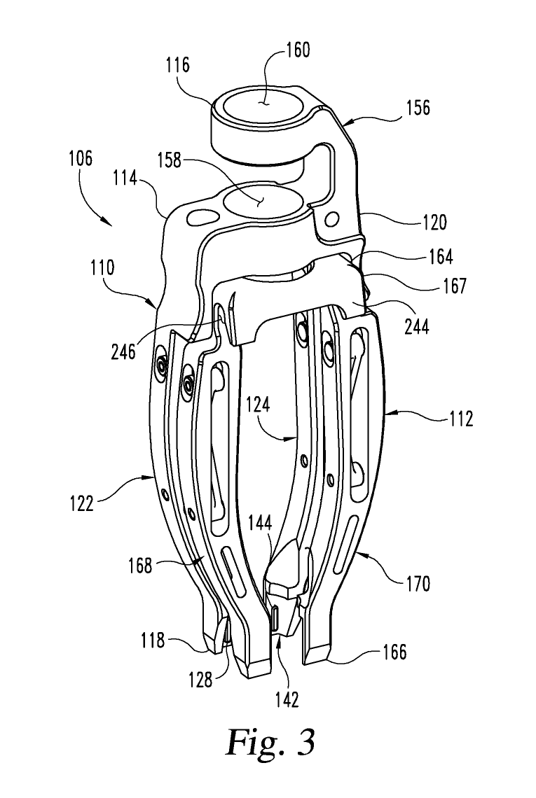

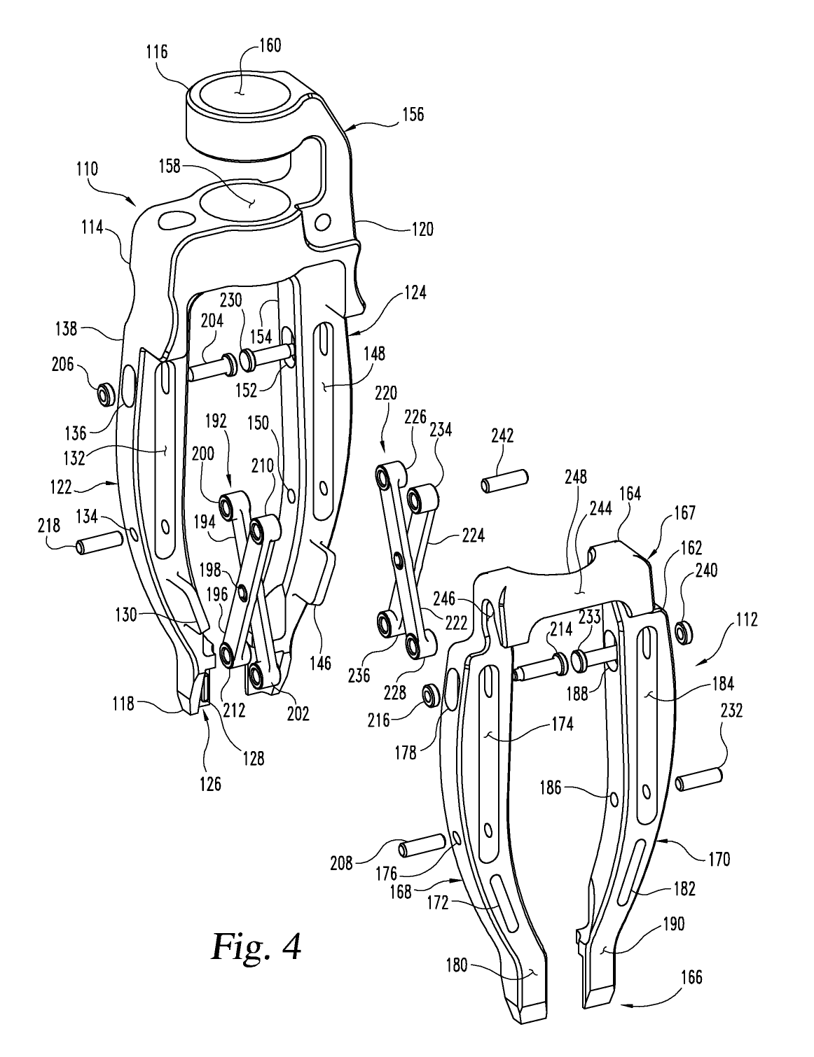

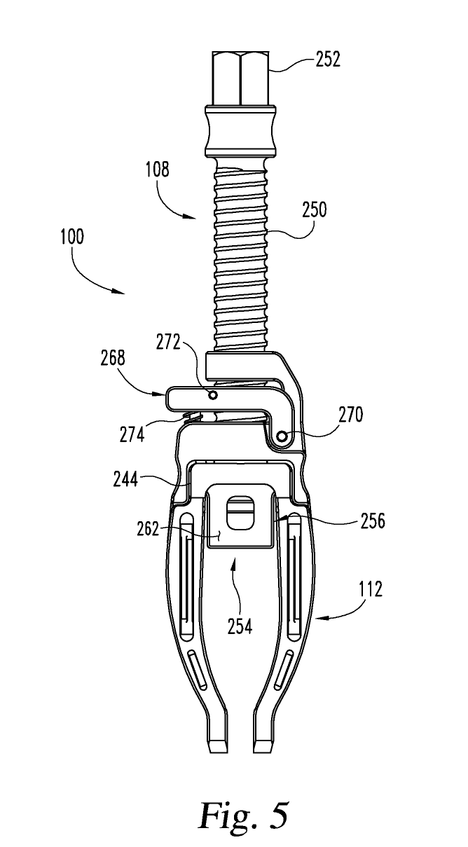

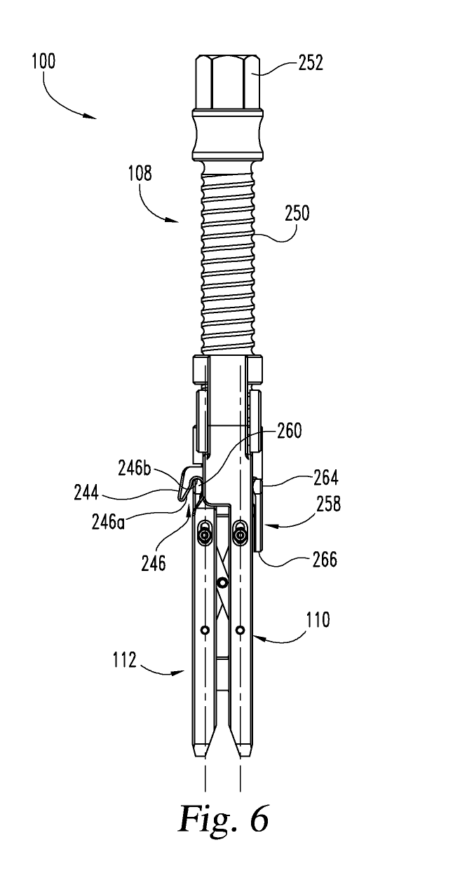

A surgical instrument 100 used to position the connecting element 16 in a desired position relative to one or more of the bone anchors 14 of the spinal implant system 10 is shown in a perspective view in FIG. 2. The instrument 100 generally extends between a proximal end 102 and an opposite distal end 104 and includes an anchor engaging portion 106 and an actuation member 108 axially displaceable relative to the anchor engaging portion 106, further details of which will be provided below. Referring collectively to FIGS. 3 and 4, further details regarding the anchor engaging portion 106 will be provided. More specifically, the anchor engaging portion 106 includes a first member 110 and a second member 112 laterally movable relative to the first member 110 between a first position where the second member 112 is spaced apart from the first member 110 (FIGS. 5 and 6), and a second position where the second member 112 is positioned between the first member 110 and the first position (FIGS. 7-10). In the illustrated form, when the second member 112 is in the second position, it is generally positioned adjacent to the first member 110 with a small gap positioned therebetween, although forms in which the gap is larger or the second member 112 abuts the first member 110 when it is in the second position are also contemplated. It should be understood that the first and second members 110, 112 generally extend substantially or entirely parallel to one another when the second member 112 is in the first and second positions relative to the first member 110.

The first member 110 includes an elongate body 114 that extends between a proximal end 116 and a distal end 118 configured to engage with the receiver member 18 of the bone anchor 14. The elongate body 114 also includes a proximal portion 120 from which first and second spaced apart arms 122, 124 distally extend. The first arm 122 includes a distal receptacle 126 that includes an elongate projection 128 and is structured to receive a portion of a first one of the branches 22, 24 of the receiver member 18 such that the elongate projection 128 engages with a first one of the laterally facing receptacles on the first one of the branches 22, 24. The first arm 122 also includes a transverse tab 130 extending therefrom toward the second member 112, and an elongate slot 132 positioned proximal of the transverse tab 130. A bore 134 extends transversely through the first arm 122 and in communication with the elongate slot 132. Additionally, an elongate slot 136 is positioned proximal of the bore 134 and extends transversely through the first arm 122 and in communication with the elongate slot 132. The first arm 122 also includes a lateral surface 138 facing away from the second member 112.

The second arm 124 includes a distal receptacle 142 that includes an elongate projection 144 and is structured to receive a portion of the first one of the branches 22, 24 of the receiver member 18 such that the elongate projection 144 engages with a second one of the laterally facing receptacles on the first one of the branches 22, 24. The second arm 124 also includes a transverse tab 146 extending therefrom toward the second member 112, and an elongate slot 148 positioned proximal of the transverse tab 146. A bore 150 extends transversely through the second arm 124 and in communication with the elongate slot 148. Additionally, an elongate slot 152 is positioned proximal of the bore 150 and extends transversely through the second arm 124 and in communication with the elongate slot 148. The second arm 124 also includes a lateral surface 154 facing away from the second member 112.

While not previously discussed, it should also be appreciated that the first member 110 also includes a proximal mounting portion 156 and apertures 158, 160 which collectively facilitate coupling of the actuation member 108 with the bone anchor engaging portion 106, further details of which will be provided below.

The second member 112 includes an elongate body 162 that extends between a proximal end 164 and a distal end 166 configured to engage with the anchor receiver member 18 of the bone anchor 14. The elongate body 162 also includes a proximal portion 167 from which third and fourth spaced apart arms 168, 170 distally extend. While not illustrated, it should be understood that the third arm 168 includes a distal receptacle that includes an elongate projection similar to the corresponding features described above in connection with the first and second arms 122, 124 of the first member 112. Similarly, the distal receptacle of the third arm 168 is structured to receive a portion of a second one of the branches 22, 24 of the receiver member 18 such that its elongate projection engages with a first one of the laterally facing receptacles on the second one of the branches 22, 24. The third arm 168 also includes an elongate slot 172 extending transversely therethrough and configured to receive the transverse tab 130 of the first arm 122. Another elongate slot 174 is positioned proximal of the elongate slot 172, and a bore 176 extends transversely through the third arm 168 and in communication with the elongate slot 174. Additionally, an elongate slot 178 is positioned proximal of the bore 176 and extends transversely through the third arm 168 and in communication with the elongate slot 174. The third arm 168 also includes a lateral surface 180 facing away from the first member 110.

While not illustrated, it should be understood that the fourth arm 170 includes a distal receptacle that includes an elongate projection similar to the corresponding features described above in connection with the first and second arms 122, 124 of the first member 112. Similarly, the distal receptacle of the fourth arm 170 is structured to receive a portion of the second one of the branches 22, 24 of the receiver member 18 such that its elongate projection engages with a second one of the laterally facing receptacles on the second one of the branches 22, 24. The fourth arm 170 also includes an elongate slot 182 extending transversely therethrough and configured to receive the transverse tab 146 of the second arm 124. Another elongate slot 184 is positioned proximal of the elongate slot 182, and a bore 186 extends transversely through the fourth arm 170 and in communication with the elongate slot 184. Additionally, an elongate slot 188 is positioned proximal of the bore 186 and extends transversely through the fourth arm 170 and in communication with the elongate slot 184. The fourth arm 170 also includes a lateral surface 190 facing away from the first member 110.

The anchor engaging portion 106 also includes a first linkage assembly 192 that is pivotably coupled between the first arm 122 of the first member 110 and the third arm 168 of the second member 112. The first linkage assembly 192 includes a first member 194 and a second member 196 pivotably coupled to the first member 194 about a pivot 198. The first member 194 includes a proximal portion 200 positionable in the elongate slot 132 of the first arm 122 of the first member 110 and pivotably engageable therewith by a pin 204. The pin 204 extends through the elongate slot 136 and is retained therein by a retaining element 206. Similarly, in this arrangement, the proximal portion 200 of the first member 192 is pivotably and axially slidably coupled with the first arm 122 of the first member 110. The first member 194 of the first linkage assembly 192 also includes a distal portion 202 positionable in the elongate slot 174 of the third arm 168 of the second member 112 and pivotably engaged therewith by a pin 208 that extends through the bore 176.

The second member 196 of the first linkage assembly 192 includes a proximal portion 210 positionable in the elongate slot 174 of the third arm 168 of the second member 112 and pivotably engageable therewith by a pin 214. The pin 214 extends through the elongate slot 178 and is retained therein by a retaining element 216. Similarly, in this arrangement, the proximal portion 210 of the second member 196 is pivotably and axially slidably coupled with the third arm 168 of the second member 112. The second member 196 of the first linkage assembly 192 also includes a distal portion 212 positionable in the elongate slot 132 of the first arm 122 of the first member 110 and pivotably engaged therewith by a pin 218 that extends through the bore 134.

The anchor engaging portion 106 also includes a second linkage assembly 220 that is pivotably coupled between the second arm 124 of the first member 110 and the fourth arm 170 of the second member 112. The second linkage assembly 220 includes a first member 222 and a second member 224 pivotably coupled to the first member 222 about a pivot (not shown). The first member 222 includes a proximal portion 226 positionable in the elongate slot 148 of the second arm 124 of the first member 110 and pivotably engageable therewith by a pin 230. The pin 230 extends through the elongate slot 152 and is retained therein by a retaining element (not shown). In this arrangement, the proximal portion 226 of the first member 222 is pivotably and axially slidably coupled with the second arm 124 of the first member 110. The first member 222 of the second linkage assembly 220 also includes a distal portion 228 positionable in the elongate slot 184 of the fourth arm 170 of the second member 112 and pivotably engaged therewith by a pin 232 that extends through the transverse bore 186.

The second member 224 of the second linkage assembly 220 includes a proximal portion 234 positionable in the elongate slot 184 of the fourth arm 170 of the second member 112 and pivotably engageable therewith by a pin 238. The pin 238 extends through the elongate slot 188 and is retained therein by a retaining element 240. In this arrangement, the proximal portion 234 of the second member 224 is pivotably and axially slidably coupled with the fourth arm 170 of the second member 112. The second member 224 of the second linkage assembly 220 also includes a distal portion 236 positionable in the elongate slot 148 of the second arm 124 of the first member 110 and pivotably engaged therewith by a pin 242 that extends through the bore 150.

The first and second linkage assemblies 192, 220 are generally laterally adjustable between a first, laterally expanded configuration when the second member 112 is in its first position relative to the first member 110 (FIGS. 5-6), and a second, laterally unexpanded configuration when the second member 112 is in its second position relative to the first member 110 (FIGS. 7-10). It should be understood that the arrangement of the first and second linkage assemblies 192, 220, and their engagement with the first and second members 110, 112, allows the second member 112 to be moved toward and away from the first member 110 while it continues to extend substantially or entirely parallel to the first member 110. In the illustrated form, the first and second members 110, 112 extend substantially or entirely parallel to one another when the second member 112 is in the first and second positions relative to the first member 110 and as the second member 112 is moved between the first and second positions.

It should be understood that receipt of the transverse tabs 130, 146 in the slots 172, 182 may prevent or reduce torsion of the anchor engaging portion 106 during reduction of the connecting element 16 with the instrument 100. Additionally, it should be further understood that while the anchor engaging portion 106 is illustrated with the first and second linkage assemblies 192, 220, non-illustrated forms in which the anchor engaging portion 106 includes only one of the linkage assemblies 192, 220 coupled between the first and second members 110, 112 are also contemplated. In other non-illustrated forms, it is also contemplated that the first and second members 110, 112 each only include a single arm with a single linkage assembly coupled therewith such that the anchoring engaging portion 106 only engages with one of the branches 22, 24 of the receiver member 18. Further, it should also be understood that the elongate slots 132, 174 cooperate with one another to house at least a portion of the first linkage assembly 192 when it is in its first and second configurations, and that the elongate slots 148, 184 cooperate with one another to house at least a portion of the second linkage assembly 220 when it is in its first and second configurations.

The proximal portion 167 of the second member 112 of the anchor engaging portion 106 also includes a guide member 244 extending laterally beyond the lateral surfaces 180, 190 of the third and fourth arms 168, 170. Additionally, the proximal portion 167 includes an elongate slot 246 or guide track that generally opens in a distal direction. As best seen in FIG. 6, the elongate slot 246 includes a first portion 246a that extends obliquely toward the first member 110 in a proximal direction, and a second portion 246b positioned proximal of the first portion 246a and extending substantially parallel with the first member 110. As will be discussed in greater detail below, the guide member 244 and the elongate slot 246 are generally configured to cooperate with a portion of the actuation member 108 to move the second member 112 between its first and second positions relative to the first member 110 upon movement of the actuation member 108 relative to the anchor engaging portion 106, further details of which will be provided below. The proximal portion 167 also includes an arcuate notch 248 configured to provide clearance for the actuation member 108 when the second member 112 is in the second position relative to the first member 110.

Referring collectively to FIGS. 2 and 5-8, further details regarding the actuation member 108 will now be provided. More particularly, in the illustrated form, the actuation member 108 is configured for moving the second member 112 between its first and second positions relative to the first member 110 to facilitate releasable engagement of the anchor engaging portion 106 with the anchor 14, and for engaging with the connecting element 16 to reduce the connecting element 16 into the receiver member 18 of the anchor 14. However, in other non-illustrated forms, it should be understood that the surgical instrument 100 may be provided with a reduction member that is separate from the actuation member 108, such that the actuation member 108 is only configured for moving the second member 112 between its first and second positions relative to the first member 110 and the reduction member is configured to engage with and reduce the connecting element 16 into the receiver member 18 of the anchor 14. In this arrangement, the actuation member 108 could be displaced relative to the anchor engaging portion 106 to facilitate engagement of the surgical instrument 100 with the anchor 14, followed by reduction of the connecting element 16 into the receiver member 18 of the anchor 14 by the separate reduction member.

Turning again to the illustrated form of the actuation member 108, it includes an externally threaded elongate shaft 250 that extends between a proximal portion 252 and a distal engaging member 254. In the illustrated form, the proximal portion 252 is provided with a plurality of flats to facilitate engagement of the actuation member 108 with an instrument having a correspondingly shaped engaging portion. Additionally, the actuation member 108 includes an elongate bore 256 extending through the proximal portion 252 and the elongate shaft 250 and opening through the distal engaging member 254 to facilitate passage of a set screw 30 and related driver through the actuation member 108 in order to secure the connecting element 16 in the channel 26 of the receiver member 18.

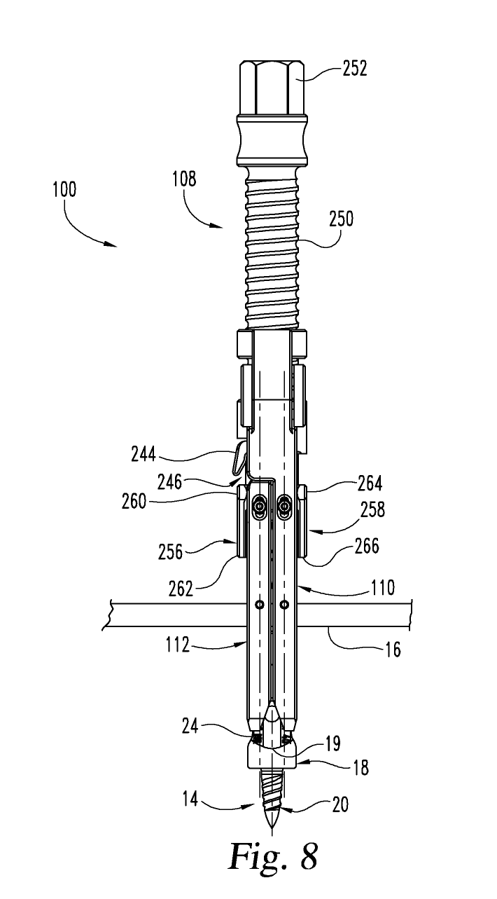

The elongate shaft 250 is pivotably coupled with the distal engaging member 254 such that the elongate shaft 250 can be rotated relative to the distal engaging member 254. In the illustrated form, the distal engaging member 254 includes a first member 256 spaced apart from a second member 258 (FIG. 8). The first member 256 includes a proximal portion 260 that laterally extends beyond a distal portion 262, and the second member 258 includes a proximal portion 264 that laterally extends beyond a distal portion 266 similar to the proximal and distal portions 260, 262 of the first member 256.

In the illustrated form, the instrument 100 also includes an engaging member 268 pivotably mounted to the first member 110 of the anchor engaging portion 106 about a pivot 270. The engaging member 268 includes an engaging portion 272 configured to engage with the external threading of the elongate shaft 250. Additionally, a biasing member 274 extends between the proximal portion 120 of the first member 110 and the engaging member 268 such that the engaging member 268 is normally biased in a proximal direction with the engaging portion 272 engaged with the elongate shaft 250. When engaged with the engaging member 268, rotation of the elongate shaft 250 in opposite directions relative to the anchor engaging portion 106 distally advances or proximally withdraws the actuation member 108. The engaging member 268 is also generally configured to facilitate distal movement of the actuation member 108 relative to the anchor engaging portion 106 without rotation of the elongate shaft 250 when it is engaged with the engaging member 268, and to prevent proximal movement of the actuation member 108 relative to the anchor engaging portion 106 without rotation of the elongate shaft 250 when it is engaged with the engaging member 268. However, the engaging member 268 may also be distally depressed to displace it from engagement with the actuation member 108, in which case the actuation member 108 may be readily displaced relative to the anchor engaging portion 106 in both proximal and distal directions without rotation.

Referring collectively to FIGS. 5-10, further details regarding the operation of the instrument 100 will be provided. More specifically, in FIGS. 5-6, the actuation member 108 is positioned in a proximal most position relative to the anchor engaging portion 106. In this configuration, the proximal portion 260 of the first member 256 of the distal engaging member 254 is positioned in the proximal portion 246b of the elongate slot 246 of the second member 112. Additionally, the proximal portion 264 of the second member 258 of the distal engaging member 254 is engaged with the lateral surfaces 138, 154 of the first and second arms 122, 124 of the first member 110. In this configuration, the second member 112 is in its first position relative to the first member 110 such that the anchor 14 can be received between the first and second members 110, 112, and the first and second linkage assemblies 192, 220 are generally in their first, laterally expanded configuration.

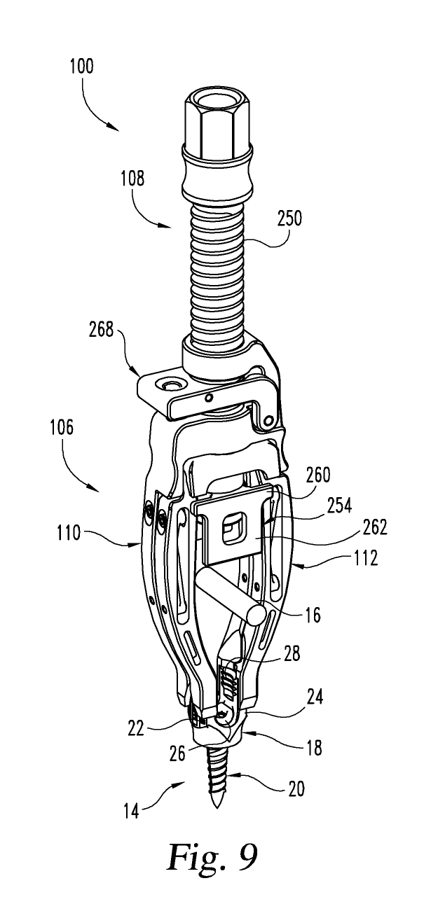

As the actuation member 108 is moved distally relative to the anchor engaging portion 106, the proximal portion 260 of the first member 256 engages with a distal surface in the first portion 246a of the elongate slot 246, which in turns forces the second member 112 toward the first member 110 in a parallel fashion as discussed above. Upon sufficient distal movement of the actuation member 108, the proximal portion 260 of the first member 256 exits the elongate slot 246 and engages with the lateral surfaces 180, 190 of the third and fourth arms 168, 170 of the second member 112 of the anchor engaging portion 106. In this configuration, the second member 112 is positioned in its second position relative to the first member 110 such that the anchor 14 is engaged by the anchor engaging portion 106, as illustrated in FIGS. 7-10, and the first and second linkage assemblies 192, 220 are generally in their second, laterally unexpanded configurations. Additionally, as illustrated in FIG. 9, the first and second members 110, 112 also extend substantially parallel to a central axis 19 of the receiver member 18 when the anchor engaging portion 106 is engaged with the anchor 14. It should also be understood that the second member 112 may also extend substantially parallel to the central axis 19 as it is moved between its first and second positions relative to the first member 110.

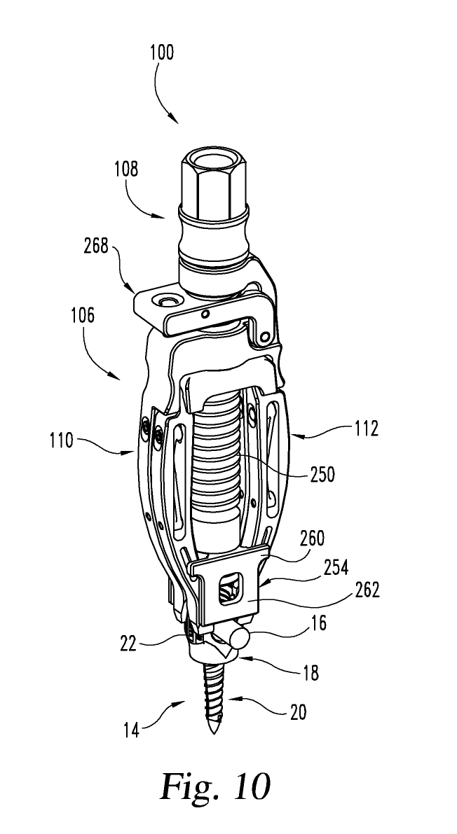

Distal movement of the actuation member 108 can continue with the proximal portions 260, 264 of the first and second members 256, 258 of the distal engaging member 254 being guided along the lateral surfaces 138, 154 and 180, 190 of the first and second members 110, 112, respectively, until the distal engaging member 254 engages with and seats the connecting element 16 in the channel 26 of the receiver member 18 (FIG. 10). Once the connecting element 16 is seated in the channel 26, a set screw 30 and related driver can be advanced through the elongate bore 256 of the actuation member 108 and engaged with the anchor 14 in order to secure the connecting element 16 in the channel 26.

Once the connecting element 16 has been properly secured in the channel 26, the actuation member 108 can be proximally displaced relative to the anchor engaging portion 106. As the actuation member 108 is proximally displaced relative to the anchor engaging portion 106, the proximal portions 260, 264 of the first and second members 256, 258 of the distal engaging member 254 are guided along the lateral surfaces 138, 154 and 180, 190 of the first and second members 110, 112, respectively, until the proximal portion 260 of the first member 256 engages with the guide member 244 and is directed into the first portion 246a of the elongate slot 246. More specifically, the proximal portion 260 of the first member 256 engages with a proximal surface in the first portion 246a of the elongate slot 246, which in turns forces the second member 112 away the first member 110 in a parallel fashion as discussed above. Upon sufficient proximal movement of the actuation member 108, the proximal portion 260 of the first member 256 is returned to the second portion 246b of the elongate slot 246. In this configuration, the second member 112 is returned to its first position relative to the first member 110 such that the anchor 14 is released by the anchor engaging portion 106, and the first and second linkage assemblies 192, 220 are generally returned to their first, laterally expanded configurations.

The instruments, devices, apparatuses, systems and methods described herein also have application with other types of instruments and implants, and may be used in other portions of the body besides the spine. The instruments, devices, apparatuses, systems and methods described herein may also be used in surgical procedures involving animals, or in demonstrations for training, education, marketing, sales and/or advertising purposes. In addition, the instruments, devices, apparatuses, systems and methods may also be used on or in connection with a non-living subject such as a cadaver, training aid or model, or in connection with testing of surgical systems, surgical procedures, orthopedic devices and/or apparatus.

Any theory, mechanism of operation, proof, or finding stated herein is meant to further enhance understanding of the present invention and is not intended to make the present invention in any way dependent upon such theory, mechanism of operation, proof, or finding. It should be understood that while the use of the word preferable, preferably or preferred in the description above indicates that the feature so described may be more desirable, it nonetheless may not be necessary and embodiments lacking the same may be contemplated as within the scope of the invention, that scope being defined by the claims that follow. In reading the claims it is intended that when words such as “a,” “an,” “at least one,” “at least a portion” are used there is no intention to limit the claim to only one item unless specifically stated to the contrary in the claim. Further, when the language “at least a portion” and/or “a portion” is used the item may include a portion and/or the entire item unless specifically stated to the contrary.

While the invention has been illustrated and described in detail in the drawings and foregoing description, the same is to be considered as illustrative and not restrictive in character, it being understood that only the selected embodiments have been shown and described and that all changes, modifications and equivalents that come within the spirit of the invention as defined herein or by any of the following claims are desired to be protected.

What is claimed is:

1. A reduction instrument, comprising:
a first member extending between a proximal end and an opposite distal end including a first anchor engagement portion;
a second member extending between a proximal end and an opposite distal end including a second anchor engagement portion, said second member being laterally movable relative to said first member between a first position spaced apart from said first member and a second position between said first position and said first member;
an actuation member axially displaceable relative to said first and second members; and
wherein said second member is moved between said first and second positions upon displacement of said actuation member relative to said first and second members, said second member extending substantially parallel to said first member during movement between said first and second positions.

2. The reduction instrument of claim 1, wherein said second member extends substantially parallel to said first member in said first and second positions.

3. The reduction instrument of claim 1, further comprising a pair of linkage assemblies positioned between and coupled to said first and second members.

4. The reduction instrument of claim 3, wherein each of said linkage assemblies comprises a first arm pivotably coupled to each of said first and second members and a second arm pivotably coupled to said first arm and each of said first and second members.

5. The reduction instrument of claim 1, wherein said second member includes a lateral surface facing away from said first member and extending from said distal end to a proximal portion of said second member, said proximal portion including a guide member extending laterally beyond said lateral surface and an elongate slot.

6. The reduction instrument of claim 5, wherein said actuation member includes a distal portion structured to engage with said second member, said distal portion being positioned in said elongate slot when said second member is in said first position and engaging with said lateral surface when said second member is in said second position.

7. The reduction instrument of claim 6, wherein said distal portion engages with a distal surface of said elongate slot upon distal movement of said distal portion from said elongate slot to said lateral surface, and said distal portion engages with a proximal surface of said elongate slot upon proximal movement of said distal portion from said lateral surface into said elongate slot.

8. The reduction instrument of claim 6, wherein said distal portion of said actuation member is structured to engage with a lateral surface of said first member facing away from said second member.

9. The reduction instrument of claim 5, wherein said elongate slot includes a first portion extending obliquely toward said first member in a proximal direction and a second portion positioned proximal of said first portion and extending substantially parallel to said first member.

10. The reduction instrument of claim 1, wherein said actuation member includes a distal portion structured to engage with said second member and positionable between a proximal position and a distal position;
wherein distal movement of said distal portion from said proximal position to said distal position moves said second member from said first position to said second position; and
wherein proximal movement of said distal portion from said distal position to said proximal position moves said second member from said second position to said first position.

11. The reduction instrument of claim 1, wherein said first member includes a pair of tabs extending toward said second member and said second member includes a pair of receptacles each structured to receive a corresponding one of said tabs.

12. The reduction instrument of claim 1, wherein said first anchor engagement portion includes a first pair of oppositely positioned elongate projections extending toward said second member and said second anchor engagement portion includes a second pair of oppositely positioned elongate projections extending toward said first member.

13. A reduction instrument, comprising:
a first member extending between a proximal end and an opposite end including a first anchor engagement portion;
a second member extending between a proximal end and an opposite distal end including a second anchor engagement portion;
a first linkage assembly pivotably coupled with said first and second members, said first linkage assembly being laterally adjustable between a first configuration where said second member is in a first position spaced apart from said first member and a second configuration where said second member is in a second position between said first position and said first member;
an actuation member axially displaceable relative to said first and second members; and
wherein said first linkage assembly is moved between said first and second configurations upon displacement of said actuation member relative to said first and second members.

14. The reduction instrument of claim 13, further comprising a second linkage assembly pivotably coupled with said first and second members, said second linkage assembly being laterally adjustable between a first configuration where said second member is in said first position and a second configuration where said second member is in said second position.

15. The reduction instrument of claim 14, wherein said first member includes a first pair of elongate slots and said second member includes a second pair of elongate slots positioned opposite of said first pair of elongate slots, said first and second pairs of elongate slots cooperating to house a portion of said first and second linkage assemblies.

16. The reduction instrument of claim 14, wherein each of said linkage assemblies comprises a first arm and a second arm pivotably coupled to said first arm;
wherein a proximal portion of said first arm is pivotably and slidably coupled with said first member and a distal portion of said first arm is pivotably coupled with said second member; and
wherein a proximal portion of said second arm is pivotably and slidably coupled with said second member and a distal portion of said second arm is pivotably coupled with said first member.

17. The reduction instrument of claim 13, wherein said second member extends substantially parallel to said first member in said first and second positions and during movement between said first and second positions.

18. A method, comprising:
providing an anchor including a bone engaging portion and a receiver member having a receiving portion;
providing a reduction instrument engageable with the anchor, the reduction instrument including:
a first member extending between a proximal end and an opposite distal end including a first anchor engagement portion;
a second member extending between a proximal end and an opposite distal end including a second anchor engagement portion; and
an actuation member axially displaceable relative to the first and second members;
positioning the reduction instrument adjacent the anchor; and
distally moving the actuation member relative to the first and second members toward the anchor to displace the second member toward the first member and releasably couple the reduction instrument with the anchor, the second member extending substantially parallel to the first member as the second member is displaced toward the first member.

19. The method of claim 18, further comprising:
positioning an elongate connecting element between the first and second members of the reduction instrument;
engaging the elongate connecting element with the actuation member and reducing the elongate connecting element into the receiving portion of the anchor; and
engaging a retaining element with the receiver member to fix the elongate connecting element in the receiving portion.

20. The method of claim 19, further comprising proximally moving the actuation member relative to the first and second members away from the anchor to displace the second member away from the first member and release the anchor from the reduction instrument, the second member extending substantially parallel to the first member as the second member is displaced away from the first member.
